(19) United States
(12) Patent Application Publication
(10) Pub. No.: US 2007/0243184 A1
(43) Pub. Date: Oct. 18, 2007

(54) PROPHYLAXIS AND TREATMENT OF ENTEROCOLITIS ASSOCIATED WITH ANTI-CTLA-4 ANTIBODY THERAPY

(76) Inventors: Steven Fischkoff, Short Hills, NJ (US); Israel Lowy, Dobbs Ferry, NY (US); Stephan Targan, Santa Monica, CA (US); James Chung-Yin Yang, Silver Spring, MD (US)

Correspondence Address: BAKER BOTTS L.L.P., 30 ROCKEFELLER PLAZA, 44th Floor, NEW YORK, NY, 10112-4498, US

(21) Appl. No.: 11/557,844

(22) Filed: Nov. 8, 2006

Related U.S. Application Data
(60) Provisional application No. 60/734,881, filed on Nov. 8, 2005.

(51) Int. Cl. A61K 39/395; A61K 31/56

(52) U.S. Cl. 424/130.1; 514/44

(57) ABSTRACT

The present invention provides methods for reducing the incidence of adverse events related to immunotherapy. More specifically, the present invention provides methods for reducing the incidence of enterocolitis associated with anti-CTLA-4 antibody immunotherapy.

### RELATED APPLICATIONS

This application claims priority to U.S. Provisional application Ser. No. 60/734,881, filed on Nov. 8, 2005, the contents of which are expressly incorporated herein by reference.

### FIELD OF THE INVENTION

The present invention relates to the field of decreasing the incidence of adverse events from immunotherapy. More specifically, the present invention relates to methods for decreasing the incidence of enterocolitis associated with anti-CTLA-4 antibody immunotherapy.

### BACKGROUND

Immune-related adverse events are a frequently observed consequence of immunostimulatory antibody therapy. These immune-related adverse events, which can be severe, and even life-threatening, include autoimmune responses, such as diarrhea, enterocolitis, dermatitis, hypophysitis, panhypopituitarism, rash, pruritis, and vitiligo (see, e.g., U.S. Patent Publication No. 2004/0241169 A1).

Anti-CTLA-4 antibodies are known immunostimulatory agents (see, e.g., PCT Publication Nos. WO 01/14424 and WO 00/37504, which describe human sequence anti-human CTLA-4 antibodies). Non-human CTLA-4 antibodies have been used in the various studies. U.S. Pat. No. 5,855,887 discloses a method of increasing the response of a mammalian T cell to antigenic stimulation by combining a T cell with a CTLA-4 blocking agent. U.S. Pat. No. 5,811,097 discloses a method of decreasing the growth of non-T cell tumors by administering a CTLA-4 blocking agent. U.S. patent application Ser. Nos. 09/644,668 and 09/948,939 disclose human CTLA-4 antibodies. Each of these patents and applications is hereby incorporated by reference in their entireties.

Therapy with an immunostimulatory agent, such as an anti-CTLA-4 antibody, is associated with certain adverse events, which appear to be mediated by the immune system. For example, adverse events related to MDX-010 (see PCT Publication No. WO 01/1424) therapy appear to have an immune etiology and may be a consequence of the intrinsic biological activity of MDX-010. These adverse events may be due to a loss of tolerance to some self-antigens or an exaggerated reaction to foreign antigens (e.g., gut bacteria). Although skin adverse events are most common, the most clinically significant immune-related adverse event following MDX-010 therapy is diarrhea secondary to enterocolitis. The enterocolitis observed following MDX-010 therapy is grossly (e.g., endoscopically) and histologically similar to inflammatory bowel disease. The gross and microscopic characteristics of ulcerative colitis and Crohn's disease are well-known. See, e.g., Harrison's Principles of Internal Medicine (15^{th} ed. 2001) pp. 1681-1685. In most cases, this immune-related enterocolitis resolves with symptomatic treatment including intravenous hydration and high-dose parenteral steroids.

### Immune Breakthrough Events

As noted above, these adverse events are an expected consequence of inhibiting CTLA-4 function. Immune-mediated events are adverse events associated with drug exposure and consistent with an immune-based mechanism of action. In terms of organ system involvement, these events have primarily involved the GI tract (diarrhea and colitis) or the skin (rash and pruritis). Diarrhea due to treatment with MDX-010 ranges from mild to very severe and may become life-threatening. Most cases of diarrhea and colitis have resolved with symptomatic treatment or corticosteroid intervention without known sequelae. Upper GI tract involvement including ileitis, duodenitis, and esophagitis has been observed. Bowel wall biopsies have usually revealed a pleomorphic infiltrate, including many lymphocytes, consistent with colitis due to an immune mediated process.

To date, 6 patients in total have experienced gastrointestinal perforation or bleeding requiring colectomy following treatment with MDX-010. Two of these patients had melanoma (representing 0.6% of all patients with melanoma enrolled in MDX-010 related protocols), while four patients had renal cell carcinoma (representing 7% of all patients with renal cell carcinoma enrolled in MDX-010 related protocols). One of the patients with melanoma also received concomitant dacarbazine. After developing diarrhea, he initially appeared to improve on intravenous steroids, but his symptoms worsened after he was tapered off the steroids, and stool cultures were positive for Clostridium difficile, requiring aggressive medical treatment. The patient also developed laboratory evidence of disseminated intravascular coagulopathy (DIC), and required plasma and platelet transfusions. Because of the refractory colitis, the patient underwent a colectomy; pathologic examination of the excised colon revealed vasculitis. The patient's post-operative course was complicated by depression and malnutrition without evidence of systemic vasculitis. The patient developed pneumonia and subsequently died. Autopsy revealed bilateral lobar pneumonia with gram positive diplococci, as well as widespread invasive aspergillus that likely contributed to the patient's complicated hospital course, GI vasculitis, and death.

In the renal study (MDX010-011), one patient had GI bleeding, which was treated with a colectomy. The bleeding developed after a single dose of MDX-010. The other 3 events in the MDX010-011 study occurred after the patients received 4 to 6 doses of MDX-010. One patient with a bowel perforation was successfully treated with a colectomy and an ostomy. Subsequently, the bowel was re-functionalized. The patient was taken off steroids and has maintained a partial response to his malignancy. The second patient with a bowel perforation exhibited no symptoms of diarrhea, but instead had constipation thought to be the result of narcotic therapy for spinal stenosis. The diagnosis of colitis was only made on autopsy. The third patient with a bowel perforation continued to have diarrhea and was intermittently treated with steroids. Upon the diagnosis of a non-catastrophic perforation, the patient declined surgical intervention based on the overall progression of disease. The patient opted for hospice care and ultimately died. The fourth patient developed symptoms of colitis after a single dose of MDX-010, had only intermittent steroid treatment, and ultimately underwent a colectomy for uncontrolled bleeding. Of the 3 patients who received steroid therapy, the initiation of steroid therapy was delayed due to poor patient reporting of symptoms to the investigator, and the therapy was compromised by patient non-compliance with the recommended treatment.

There have been no reported gastrointestinal perforations or colectomies in patients with breast or prostate cancer. The overall incidence of gastrointestinal perforations and/or colectomies is less than 2% of patients.

Skin toxicity in patients receiving MDX-010 has manifested as rash and pruritis, and, when biopsied, pleomorphic infiltrates have been noted in the skin. Some patients have developed vitiligo associated with MDX-010 administration. In our studies, there have been 7 cases of hypopituitarism reported to date, presumably due to immune-mediated hypophysitis. Corticosteroid treatment, either as replacement therapy or as high-dose therapy, has resulted in resolution of clinical symptoms. The effect of high-dose corticosteroid therapy on reversing pituitary abnormalities is unknown. Ocular inflammation, specifically Grade 2 or Grade 3 episcleritis or uveitis, has been reported in 6 patients; it has occurred in conjunction with GI symptoms in 4 of these patients. In addition, primary adrenal insufficiency has been noted in 3 patients. One case each of autoimmune meningitis and granulomatous tubulointerstitial nephritis has been associated with MDX-010 (BMS-734016) administration.

With the exception of the cases requiring colectomy, these autoimmune-like adverse events have been readily manageable and reversible with supportive care or corticosteroid treatment.

Interestingly, in one of our studies, almost 45% of the patients developing an autoimmune-like adverse event have also experienced a clinical response, including a patient with hypopituitarism, who demonstrated a durable complete response. These adverse events, likely reflect a loss of tolerance to some self antigens, or a hyper-response to bacterial antigens present in the gut or skin, and are therefore mechanism-related and may be directly linked to the clinical antitumor activity of MDX-010.

Accordingly, it would be desirable to provide methods for effective treatment of diseases or conditions with immunostimulatory antibodies, e.g., antibodies to CTLA-4, which decrease the incidence and/or severity of an immune-related adverse event. In particular, a need exists for prophylactic treatment of immune-related enterocolitis following immunostimulatory therapeutic antibody treatment, which does not interfere with the desired immune enhancement (e.g., anti-tumor immunity).

### SUMMARY OF THE INVENTION

The present invention advantageously provides a method for reducing the incidence of enterocolitis induced by an immunostimulatory therapeutic antibody in a patient through the administration of an effective amount of a non-absorbable steroid to the patient in conjunction with administration of the immunostimulatory therapeutic antibody. In a specific embodiment, the immunostimulatory therapeutic antibody is an anti-CTLA-4 antibody.

The methods and compositions of the present invention provide for decreasing the incidence of immunostimulatory therapeutic antibody-induced enterocolitis, in turn permitting a greater number of patients to complete immunotherapy; permitting a higher dose or greater frequency of administration because therapy limiting enterocolitis is avoided; and avoiding any adverse effect on the anti-tumor effect of the antibody due to the immunosuppressive effect of systemic steroids.

Thus, the invention relates in one embodiment to a method for reducing the incidence of enterocolitis induced by an immunostimulatory therapeutic antibody in a patient, which method comprises administering an effective amount of a non-absorbable steroid to the patient.

A particular advantage of the invention results from a method for reducing the inflammation of the gastrointestinal tract induced by an immunostimulatory therapeutic antibody in a patient. In some instances, administration of the therapeutic antibody can lead to inflammation of the gastrointestinal tract which results in diarrhea. The method of the present invention comprises administering an effective amount of a non-absorbable steroid to the patient in order to decrease the incidence of enterocolitis induced by an immunostimulatory therapeutic antibody in a patient.

A non-absorbable steroid can be administered orally, rectally or orally and rectally. In yet a further embodiment, the invention provides for co-administering a salicylate with the non-absorbable steroid. A particular non-absorbable steroid suitable for use in all embodiments of the invention is budesonide.

In a further aspect of the foregoing methods, the antibody is an anti-CTLA-4 antibody, particularly a human sequence antibody that binds to human CTLA-4. In specific examples described herein, the anti-CTLA-4 antibody is antibody 10D1 (MDX-010; ipilimumab).

In another embodiment, the invention provides a method for increasing a dose or frequency of administration, or both, of a therapeutic anti-CTLA-4 antibody administered to a patient. This method comprises administering a first dose of a non-absorbable steroid to the patient with a first dose of the anti-CTLA-4 antibody, followed by maintaining the patient on a dosage regimen of the non-absorbable steroid during the period when the patient receives additional doses of the anti-CTLA-4 antibody. Administration of the non-absorbable steroid to the patient can be continued following completion of the course of anti-CTLA-4 antibody therapy to further inhibit any potential development of enterocolitis. In a particular embodiment, administration of the non-absorbable steroid is continued for about 6 weeks following completion of the course of anti-CTLA-4 antibody therapy. In another particular embodiment involving increased frequency of dosing with the anti-CTLA-4 antibody, the anti-CTLA-4 antibody is administered more frequently than once every 4 weeks. In another specific embodiment, the amount of anti-CTLA-4 antibody administered in any single dose is greater than about 3 mg/kg, i.e., the usual dose of anti-CTLA-4 antibody administered without prophylaxis. In these aspects of the invention, the anti-CTLA-4 antibody can be a human sequence antibody that binds to human CTLA-4. In specific examples described herein, the anti-CTLA-4 antibody is antibody 10D1 (MDX-010; ipilimumab).

In a specific embodiment, the invention provides a method for reducing the incidence of enterocolitis induced by an anti-CTLA-4 antibody in a patient. This method comprises administering 10 mg/kg of the anti-CTLA-4 antibody intravenously to the patient at weeks 1, 4, 7 and 10, and administering 9 mg of budesonide to the patient with a first dose of anti-CTLA-4 antibody. In a further embodiment, the invention includes continuing budesonide administration to the patient at a dose of 9 mg/day until week 8. In yet a further embodiment, the invention includes administering 6 mg/day of budesonide to the patient from week 8 until week 12.

All aspects of the invention pertain to any therapeutic administration of an immunostimulatory antibody, particularly an anti-CTLA-4 antibody. In specific embodiments, the anti-CTLA-4 antibody is administered for the treatment of malignant melanoma, prostate cancer or ovarian cancer.

### DETAILED DESCRIPTION

As used herein, an “immunostimulatory therapeutic molecule” is any molecule (e.g., small molecule, protein, peptide, nucleic acid molecule, or antibody) that is administered to a patient to stimulate the patient's immune system for the purpose of treating a disease (e.g., a cancer or infectious disease). As used herein, an “immunostimulatory therapeutic antibody” is a subset of an immunostimulatory therapeutic molecule and is any antibody that is administered to a patient to stimulate the patient's immune system for the purpose of treating a disease (e.g., a cancer or infectious disease). In particular, an immunostimulatory therapeutic antibody of the invention relates to an anti-CTLA-4 antibody. In a specific embodiment, the antibody is specific for human CTLA-4. In a further embodiment, the antibody is a human sequence antibody, e.g., antibody 10D1 as disclosed in PCT Publication No. WO 01/14424. Other immunostimulatory therapeutic antibodies according to the present invention include, for example, anti-PD-1 antibodies and anti-BTLA antibodies.

As used herein, “enterocolitis” is an inflammatory condition of the colon (i.e., the large intestine) and/or small intestine that can be associated with symptoms such as diarrhea, cramping, abdominal pain, bloating and/or constipation; or signs such as a bowel (e.g., colon) wall that is edematous, hyperemic, and/or friable (as observed, for example, during an endoscopic examination).

As used herein, “enterocolitis induced by an immunostimulatory therapeutic antibody” means an enterocolitis that: (1) has its first occurrence in a patient concurrent with, or shortly after (i.e., days or weeks), administration of an immunostimulatory therapeutic antibody, and (2) is identified as an enterocolitis induced by an immunostimulatory therapeutic antibody by a physician, or (3) is not identified as an enterocolitis of another etiology (e.g., Clostridium difficile toxin) by a physician.

Except when noted, the terms “patient” or “subject” are used interchangeably and refer to mammals such as human patients and non-human primates, as well as experimental animals such as rabbits, rats, and mice, and other animals. Animals include all vertebrates, e.g., mammals and non-mammals, such as sheep, dogs, cows, chickens, amphibians, and reptiles. Usually such patient is receiving an immunostimulatory antibody, e.g., an anti-CTLA-4 antibody, to treat a disease or condition. PCT Publication No. WO 01/14424 sets forth diseases and conditions treatable with an anti-CTLA-4 antibody, including but not limited to malignant melanoma, prostate cancer, and ovarian cancer. The present specification incorporates by reference the subject matter disclosed in PCT Publication No. WO 01/14424 relating to disease treatment.

The terms “to reduce the incidence of enterocolitis” and “decrease the incidence of enterocolitis” mean lowering the rate of occurrence of enterocolitis induced by an immunostimulatory therapeutic antibody in patients who are administered a non-absorbable steroid according to the methods of the present invention relative to the rate of occurrence of such an enterocolitis in patients who are not administered a non-absorbable steroid.

The terms “cytotoxic T lymphocyte-associated antigen-4,” “CTLA-4,” “CTLA4,” “CTLA-4 antigen” and “CD152” (see, e.g., Murata, Am. J. Pathol. 1999;155:453-460) are used interchangeably, and include variants, isoforms, species homologs of human CTLA-4, and analogs having at least one common epitope with CTLA-4 (see, e.g., Balzano (1992) Int. J. Cancer Suppl. 7:28-32). The complete sequence of CTLA-4 is found in GenBank Accession No. L15006.

The phrase “immune cell response” refers to the response of immune system cells to external or internal stimuli (e.g., antigen, cytokines, chemokines, and other cells) producing biochemical changes in the immune cells that result in immune cell migration, killing of target cells, phagocytosis, production of antibodies, other soluble effectors of the immune response, and the like.

The term “immune response” refers to the concerted action of lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues.

### MDX-010 Therapy

The human monoclonal antibody MDX-010 (Medarex, Inc.) in clinical development corresponds to monoclonal antibody 10D1, which is disclosed in U.S. Patent Publication No. 2005/0201994, PCT Publication No. WO 01/14424, U.S. Pat. No. 6,984,720, and U.S. Patent Publication No. 2002/086014. MDX-101 is also referred to as ipilimumab. MDX-010 has been administered as single or multiple doses, alone or in combination with a vaccine, chemotherapy, or interleukin-2 to greater than 500 patients diagnosed with metastatic melanoma, prostate cancer, lymphoma, renal cell cancer, breast cancer, ovarian cancer, and HIV.

Other anti-CTLA-4 antibodies that can be used in a method of the present invention include, for example, those disclosed in: WO 98/42752; WO 00/37504; U.S. Pat. No. 6,682,736; U.S. Pat. No. 6,207,156; Hurwitz et al., PNAS 1998;95(17):10067-10071; Camacho et al., J Clin Oncology 2004:22(145):abstract no. 2505 (antibody CP-675206); and Mokyr, et al., Cancer Research 1998;58:5301-5304.

The dosage and schedule for administration of an anti-CTLA-4 antibody used in a method of the present invention can be determined by one of skill in the art. For example, the dosage of the antibody can range from about 0.1 mg/kg to about 50 mg/kg, typically from about 1 mg/kg to about 25 mg/kg. In particular embodiments, the anti-CTLA-4 antibody dosage is 1 mg/kg, 3 mg/kg, 5 mg/kg, 10 mg/kg, 15 mg/kg, 20 mg/kg or 25 mg/kg. The dosage schedule for administration of the antibody can vary depending on the desired aggressiveness of the therapy, as determined by the practitioner. Dosages and dosage schedules are described in U.S. Patent Publication No. 20020086014. In a specific embodiment, the dosage of anti-CTLA-4 antibody is 10 mg/kg.

### Enterocolitis Associated With Anti-CTLA-4 Antibody Therapy

Organs that most commonly exhibit immune-related adverse events following anti-CTLA-4 antibody therapy are the GI tract (e.g., diarrhea and colitis) and the skin (e.g., rash and pruritis). Diarrhea following MDX-010 treatment can range from mild to severe, and can even be life-threatening. Colonic wall biopsies in patients with post-MDX-010 diarrhea have revealed pleomorphic infiltrates, which include many lymphocytes and are consistent with colitis due to an immune-mediated process. Most cases of diarrhea and colitis resolve with symptomatic treatment (e.g., fluid replacement) or corticosteroid treatment.

Non-colonic gastrointestinal immune-related adverse events have also been observed in the esophagus (esophagitis), duodenum (duodenitis), and ileum (ileitis).

The present invention provides methods for reducing the incidence of immunostimulatory therapeutic antibody-induced enterocolitis and/or diarrhea by administering a non-absorbable steroid to the patient. Because any patient who will receive an immunostimulatory therapeutic antibody is at risk for developing enterocolitis and/or diarrhea induced by such an antibody, this entire patient population is suitable for therapy according to the methods of the present invention.

Although steroids have been administered to treat inflammatory bowel disease (IBD) and prevent exacerbations of IBD, they have not been used to prevent (decrease the incidence of) IBD in patients who have not been diagnosed with IBD. The significant side effects associated with steroids, even non-absorbable steroids, have discouraged such use prophylactically.

### Non-Absorbable Steroids

The present invention encompasses administration of any non-absorbable steroid in conjunction with an immunostimulatory therapeutic antibody. As used herein, a “non-absorbable steroid” is a glucocorticoid that exhibits extensive first pass metabolism such that, following metabolism in the liver, the bioavailability of the steroid is low, i.e., less than about 20%, preferably less than about 15%.

### Budesonide

In one embodiment of the invention, the non-absorbable steroid is budesonide. Budesonide is a locally-acting glucocorticosteroid, which is extensively metabolized, primarily by the liver, following oral administration. ENTOCORT EC® (Astra-Zeneca) is a pH- and time-dependent oral formulation of budesonide developed to optimize drug delivery to the ileum and throughout the colon. ENTOCORT EC® is approved in the U.S. for the treatment of mild to moderate Crohn's disease involving the ileum and/or ascending colon. The usual oral dosage of ENTOCORT EC® for the treatment of Crohn's disease is 6 to 9 mg/day. ENTOCORT EC® is released in the intestines before being absorbed and retained in the gut mucosa. Once it passes through the gut mucosa target tissue, ENTOCORT EC® is extensively metabolized by the cytochrome P450 system in the liver to metabolites with negligible glucocorticoid activity. Therefore, the bioavailability is low (about 10%). The low bioavailability of budesonide results in an improved therapeutic ratio compared to other glucocorticoids with less extensive first-pass metabolism. Budesonide results in fewer adverse effects, including less hypothalamic-pituitary suppression, than systemically-acting corticosteroids. However, chronic administration of ENTOCORT EC® can result in systemic glucocorticoid effects such as hypercorticism and adrenal suppression. See PDR 58^{th} ed. 2004; 608-610.

### Dose

One of skill in the art can readily determine the effective amount of a non-absorbable steroid to be administered according to the methods of the present invention. In general, an effective amount of a non-absorbable steroid according to the invention is the lowest amount required to produce a therapeutic effect, i.e., reduction of the incidence of enterocolitis induced by an immunostimulatory therapeutic antibody. One of skill in the art can consult the label of a non-absorbable steroid for dosing information. The exact amount to be administered to a patient can vary depending on the state and severity of the disorder and the physical condition of the patient. A non-absorbable steroid according to the invention can be administered in one daily dose or in divided doses.

In a particular embodiment of a method according to the present invention, budesonide is administered in a dosage of about 1 mg/day to about 20 mg/day, preferably in a dosage of about 3 mg/day to about 15 mg/day, and most preferably in a dosage of about 6 mg/day to about 9 mg/day.

According to the present invention, an immunostimulatory therapeutic antibody and a non-absorbable steroid can be administered concurrently (e.g., on the same day). Alternatively, according to the present invention, the first dose of a non-absorbable steroid can be administered before the first dose of an immunostimulatory therapeutic antibody or following the first dose of an immunostimulatory therapeutic antibody.

### Route of Administration

The present invention encompasses the delivery of a non-absorbable steroid (e.g., budesonide) by any route that provides direct delivery to a segment of a patient's gastrointestinal (GI) tract. Thus, oral, rectal and enteral (e.g., via an ostomy or feeding tube) routes of administration are encompassed by the present invention. The dosage form of the non-absorbable steroid can be any dosage form that permits direct delivery to the GI tract. Such dosage forms include, for example, a tablet, a capsule, oral suspension or enema.

In an embodiment of the invention, a non-absorbable steroid can be administered by more than one route to decrease the incidence of immunostimulatory therapeutic antibody-induced enterocolitis. For example, the incidence of immunostimulatory therapeutic antibody-induced enterocolitis involving the entire colon can be reduced according to the invention by administering a non-absorbable steroid both orally via a tablet and rectally via an enema. In this example, delivery of the non-absorbable steroid to the distal small intestine (ileum) and proximal large intestine (right or ascending colon, transverse colon) is ensured by the oral administration of the steroid, and delivery of the non-absorbable steroid to the distal large intestine (transverse, left or descending colon, rectum) is ensured by the rectal administration of the steroid.

### Salicylates

According to the present invention, the incidence of enterocolitis induced by an immunostimulatory therapeutic antibody can be reduced by administering a non-absorbable steroid in combination with a salicylate. Salicylates according to the present invention include 5-ASA agents such as, for example: sulfasalazine (AZULFIDINE®), Pharmacia & UpJohn); olsalazine (DIPENTUM®, Pharmacia & UpJohn); balsalazide (COLAZAL®, Salix Pharmaceuticals, Inc.); and mesalamine (ASACOL(g, Procter & Gamble Pharmaceuticals; PENTASA®, Shire US; CANASA®, Axcan Scandipharm, Inc.; ROWASA®, Solvay).

In accordance with the methods of the present invention, a salicylate administered in combination with a non-absorbable steroid includes any overlapping or sequential administration of the salicylate and the non-absorbable steroid for the purpose of decreasing the incidence of enterocolitis induced by an immunostimulatory antibody. Thus, for example, methods for reducing the incidence of enterocolitis induced by an immunostimulatory antibody according to the present invention encompasses administering a salicylate and a non-absorbable simultaneously or non-simultaneously (e.g., a salicylate is administered 6 hours after a non-absorbable steroid).

Further, according to the present invention, a salicylate and a non-absorbable steroid can be administered by the same route (e.g., both are administered orally) or by different routes (e.g., a salicylate is administered orally and a non-absorbable steroid is administered rectally).

The dosage and frequency of administration of a salicylate used in a method of the invention can be the same as the recommended dosage found on the salicylate product label, or one of skill in the art can modify the dosage or dosage schedule based on the needs of the patient.

### EXAMPLES

The present invention is also described by means of the following examples. However, the use of these or other examples anywhere in the specification is illustrative only and in no way limits the scope and meaning of the invention or of any exemplified term. Likewise, the invention is not limited to any particular preferred embodiments described herein. Indeed, many modifications and variations of the invention may be apparent to those skilled in the art upon reading this specification and can be made without departing from its spirit and scope. The invention is therefore to be limited only by the terms of the appended claims along with the full scope of equivalents to which the claims are entitled.

### Example 1

### Therapeutic MDX-010 and Prophylactic Oral Budesonide (ENTOCORT EC®) Therapy in Patients With Stage III or IV Melanoma

Study Design: This is a randomized, double-blind, placebo-controlled, Phase II study of MDX-010 (BMS-734016) administered with or without prophylactic oral budesonide (ENTOCOR EC®) in patients with previously treated, unresectable Stage III or IV melanoma.

This protocol is divided into four phases, the Screening Phase, the Induction Phase (Week 1 through week 24 tumor assessment visit), the Maintenance Phase (Week 24 dose visit through week 48), and the Follow-Up Phase.

Patients will undergo screening evaluations to determine eligibility. Once eligibility is established and patients have signed an informed consent, an optional pre-treatment tumor biopsy will be obtained, patients will be vaccinated, and Delayed Type Hypersensitivity (DTH) skin tests will be preformed. On Day 1 prior to drug administration, blood samples will be collected for baseline flow cytometry, immune cell function [Enzyme linked immunospot (ELISPOT)], markers of inflammation, mRNA expression, PK, and immunogenicity. A baseline stool sample will be collected for calprotectin and WBCs.

Dosing: Each patient will receive MDX-010 (BMS-734016) at a dose of 10 mg/kg intravenous (IV) administered as 4 single doses every three weeks (Weeks 1, 4, 7 and 10) and randomized in a double-blind fashion in a 1:1 ratio to 9 mg of oral budesonide (ENTOCORT EC®) or placebo once daily until Week 8, then to 6 mg oral budesonide (ENTOCORT EC®) or placebo once daily to Week 12 during the Induction Phase of the study. Patients will be given a 21 day supply of budesonide (ENTOCORT EC®) or placebo and will be instructed to complete a diary of drug administration and gastrointestinal symptoms. Any subject who develops Grade 2 diarrhea will discontinue budesonide/placebo and commence open-label oral budesonide (ENTOCORT EC®) 9 mg daily. Any subject who develops Grade 3 or 4 diarrhea will immediately discontinue MDX-010 (BMS-734016) and budesonide/placebo, commence IV hydration and high dose oral prednisolone or intravenous methylprednisolone, until symptoms resolve to Grade 2. During the Maintenance Phase, non-progressing patients who have not experienced unacceptable toxicity in the Induction Phase are eligible to receive additional single doses of MDX-010 (BMS-734016) every 12 Weeks (i.e. Week 24, 36, 48 in the first year) until progression, unacceptable toxicity or withdrawal of consent.

Study Assessments: Flexible sigmoidoscopy (or colonoscopy, if appropriate) with 3 to 5 colonic biopsies for processing in a standard paraffin block will be performed for all patients after the second dose of MDX-010 (BMS-734016). Any patient experiencing Grade 2 diarrhea (increase in 4-6 stools per day over baseline) will undergo a second flexible sigmoidoscopy procedure with colonic biopsy. All patients with confirmed colitis will also have an ophthalmologic examination, including slit-lamp, to rule-out uveitis.

While in the study, patients will be required to visit the investigator's office or clinic for physical examinations, vital sign measurements, ECOG performance status evaluation, toxicity assessment, laboratory safety testing, pharmacodynamic (PD) testing, periodic PK testing and administration of study drugs. Assessment of intra-tumoral immune response will be assessed by tumor biopsy 24-72 hours after the second MDX-010 (BMS-734016) dose.

Tumor Assessments: To insure a uniform tumor measurement schedule for all patients, radiological assessments (with pre-planned confirmation scans) will be performed for all patients at Week 12 with additional assessment for all non-progressing patients at Weeks 16, 20, 24 in the Induction Phase and every 6 weeks through Week 48 (i.e. Weeks 30, 36, 42 and 48) in the Maintenance Phase. In the weeks when both tumor assessments and dosing are scheduled (i.e. Weeks 24, 36 and 48) the tumor assessment will precede the pre-planned dosing and only non-progressors will receive additional maintenance doses. For non-progressors who continue dosing beyond the first year in the Maintenance Phase, tumor assessments will be done every 12 weeks (the same week as and preceding the pre-planned maintenance doses).

Of note, most responses observed to date have occurred by Week 12, even in patients with initial progression. As such, all patients who receive at least one dose of MDX-010 (BMS-734016) will, whenever possible, first return for the tumor re-staging assessment at Week 12 (and not before). After patients have been treated in the Induction Phase, they will either enter the Maintenance Phase or the Follow-up Phase, depending upon whether or not they meet eligibility criteria to enter the Maintenance Phase.

All patients who discontinue treatment due to a drug-related adverse event prior to first re-staging at Week 12 are required to return for the Week 12 visit and Week 16 (for confirmation, if non-progressing at Week 12). If such patients are found to have achieved Stable Disease or a Late Objective Response at the Week 12 and 16 tumor assessments they should, if possible, continue to be re-staged as per the protocol schedule of tumor assessments, but they cannot receive additional dosing unless they meet the criteria for Entry into the Maintenance Phase.

Duration of Study: It is anticipated that 12 months will be required to complete accrual, and the study will take 19 months to complete. The primary analysis will be performed when the last non-progressing patient has been followed to the tumor re-staging assessment at Week 26. The end of the study will occur at the same time as the primary analysis. Any patients who remain on treatment with MDX-010 (BMS-734016) at the end of the trial will be switched to a follow-up protocol to enable the current study to be closed and reported.

Test Product, Dose and Mode of Administration, Duration of Treatment: Each patient will receive MDX-010 (BMS-734016) 10 mg/kg as 4 single doses via IV infusions as tolerated at Weeks 1, 4, 7 and 10 (Induction Phase). The antibody is not to be administered as an IV push or bolus injection. Patients who are eligible for extended doses in the Maintenance Phase will receive 10 mg/kg as a single dose via IV infusion on Weeks 24, 36, 48 and every 12 weeks thereafter until unacceptable toxicity, tumor progression or consent withdrawal. In addition, nine (9) mg of oral budesonide (ENTOCORT EC®) or placebo, will be administered daily starting on Day 1 until Week 8 and then six (6) mg daily dosing until Week 12. Once off treatment, patients will continue to be followed every 3 months via telephone until death, even if they are started on additional non-protocol therapy.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Patents, patent applications, publications, product descriptions, and protocols are cited in this application are hereby incorporated by reference in their entireties for all purposes.

1. A method for reducing the incidence of enterocolitis induced by an immunostimulatory therapeutic antibody in a patient, comprising administering an effective amount of a non-absorbable steroid to the patient.

2. The method according to claim 1, wherein the non-absorbable steroid is budesonide.

3. The method according to claim 1, wherein the antibody is an anti-CTLA-4 antibody.

4. The method according to claim 3, wherein the anti-CTLA-4 antibody is a human sequence antibody that binds to human CTLA-4.

5. The method according to claim 4, wherein the anti-CTLA-4 antibody is antibody 10D1 (ipilimumab).

6. The method according to claim 1, wherein the route of administration of the non-absorbable steroid is selected from the group consisting of oral, rectal and a combination thereof.

7. The method according to claim 1, further comprising administering a salicylate.

8. The method according to claim 3, wherein the anti-CTLA-4 antibody is administered for the treatment of malignant melanoma, prostate cancer or ovarian cancer.

9. A method for increasing a dose of a therapeutic anti-CTLA-4 antibody administered to a patient, comprising:
(a) administering a first dose of a non-absorbable steroid to the patient prior to or with a first dose of the anti-CTLA-4 antibody, and
(b) maintaining the patient on a dosage regimen of the non-absorbable steroid during the period when the patient receives at least one additional dose of the anti-CTLA-4 antibody;
wherein an additional dose of the anti-CTLA-4 antibody is administered in an amount greater than the first dose of the anti-CTLA-4 antibody and/or a previously administered additional dose of the anti-CTLA-4 antibody.

10. The method according to claim 9, wherein the non-absorbable steroid is budesonide.

11. The method according to claim 9, further comprising continuing administration of the non-absorbable steroid to the patient following completion of the course of anti-CTLA-4 antibody therapy.

12. The method of claim 11, wherein administration of the non-absorbable steroid is continued for about 6 weeks following completion of the course of anti-CTLA-4 antibody therapy.

13. The method of claim 9, wherein the amount of anti-CTLA-4 antibody administered in any additional dose is greater than about 3 mg/kg.

14. The method according to claim 9, wherein the route of administration of the non-absorbable steroid is selected from the group consisting of oral, rectal and a combination thereof.

15. The method according to claim 9, further comprising administering a salicylate.

16. The method according to claim 9, wherein the anti-CTLA-4 antibody is administered for the treatment of malignant melanoma, prostate cancer or ovarian cancer.

17. A method for increasing a frequency of administration of a therapeutic anti-CTLA-4 antibody to a patient, comprising:
(a) administering to the patient a first dose of a non-absorbable steroid prior to or with a first dose of the anti-CTLA-4 antibody,
(b) maintaining the patient on a dosage regimen of the non-absorbable steroid during the period when the patient receives additional doses of the anti-CTLA-4 antibody, and
(c) continuing administration of the non-absorbable steroid to the patient following completion of the course of anti-CTLA-4 antibody therapy.

18. The method according to claim 17, wherein the non-absorbable steroid is budesonide.

19. The method of claim 17, wherein administration of the non-absorbable steroid is continued for about 6 weeks following completion of the course of anti-CTLA-4 antibody therapy.

20. The method of claim 17, wherein the anti-CTLA-4 antibody is administered more frequently than every 4 weeks.

21. The method according to claim 17, wherein the route of administration of the non-absorbable steroid is selected from the group consisting of oral, rectal and a combination thereof.

22. The method according to claim 17, further comprising administering a salicylate.

23. The method according to claim 17, wherein the anti-CTLA-4 antibody is administered for the treatment of malignant melanoma, prostate cancer or ovarian cancer.

24. A method for reducing the incidence of inflammation of the gastrointestinal tract by an immunostimulatory therapeutic antibody in a patient, comprising administering an effective amount of a non-absorbable steroid to the patient.

25. The method according to claim 24, wherein the non-absorbable steroid is budesonide.

26. The method according to claim 24, wherein the antibody is an anti-CTLA-4 antibody.

27. The method according to claim 26, wherein the anti-CTLA-4 antibody is a human sequence antibody that binds to human CTLA-4.

28. The method according to claim 27, wherein the anti-CTLA-4 antibody is antibody 10D1 (ipilimumab).

29. The method according to claim 24, wherein the inflammation of the gastrointestinal tract results in diarrhea.

30. The method according to claim 24, wherein the route of administration of the non-absorbable steroid is selected from the group consisting of oral, rectal and a combination thereof.

31. The method according to claim 24, further comprising administering a salicylate.

32. The method according to claim 26, wherein the anti-CTLA-4 antibody is administered for the treatment of malignant melanoma, prostate cancer or ovarian cancer.

33. A method for reducing the incidence of enterocolitis induced by an anti-CTLA-4 antibody in a patient, comprising:
(a) administering 10 mg/kg of the anti-CTLA-4 antibody intravenously to the patient at weeks 1, 4, 7 and 10;
(b) administering 9 mg of budesonide to the patient with a first dose of anti-CTLA-4 antibody;
(c) continuing budesonide administration to the patient at a dose of 9 mg/day until week 8; and
(d) administering 6 mg/day of budesonide to the patient from week 8 until week 12.

34. The method according to claim 33, wherein the route of administration of the budesonide is selected from the group consisting of oral, rectal and a combination thereof.

35. The method according to claim 33, further comprising administering a salicylate.

36. The method according to claim 33, wherein the anti-CTLA-4 antibody is administered for the treatment of malignant melanoma, prostate cancer or ovarian cancer.
